(19) United States
(12) Patent Application Publication
(10) Pub. No.: US 2015/0045236 A1
(43) Pub. Date: Feb. 12, 2015

(54) ACCELERATING THE DEVELOPMENT OF AGRICULTURAL PRODUCTS USING ANCHORED ENRICHMENT

(71) Applicant: The Florida State University Research Foundation, Inc., Tallahassee, FL (US)

(72) Inventor: Alan Richard Lemmon, Tallahassee, FL (US)

(21) Appl. No.: 14/524,614

(22) Filed: Oct. 27, 2014

Related U.S. Application Data
(63) Continuation-in-part of application No. 13/749,204, filed on Jan. 24, 2013.
(60) Provisional application No. 61/895,777, filed on Oct. 25, 2013.
(60) Provisional application No. 61/590,136, filed on Jan. 24, 2012.

(51) Int. Cl. C12Q 1/68

(52) U.S. Cl. CPC C12Q 1/6874
USPC 506/2

(57) ABSTRACT

Hybrid enrichment technology as applied to enhancing agricultural production, for example including, but not limited to, crop production, pesticide development, crop trait development, and pest control. The methodology pools samples prior to library production, thus reducing cost and increasing efficiency of sequencing across- and within-species targets.

### CROSS REFERENCE TO RELATED APPLICATIONS

This non-provisional application claims priority to U.S. Provisional Application No. 61/895,777, entitled “Accelerating the Development of Agricultural Products Using Anchored Enrichment”, filed Oct. 25, 2013, the entirety of which is incorporated herein by reference. This non-provisional application also is a continuation-in-part of and claims priority to co-pending U.S. Nonprovisional application Ser. No. 13/749,204, entitled “System and Method for Anchored Hybrid Enrichment”, filed Jan. 24, 2013, which claims priority to U.S. Provisional Application No. 61/590,136, entitled “Anchored Hybrid Enrichment”, filed Jan. 24, 2012, both of which are incorporated herein by reference in their entireties.

### BACKGROUND OF THE INVENTION

1. Field of the Invention

This invention relates, generally, to crop development and pest control. More specifically, it relates to use of anchored hybrid enrichment to accelerate the development of agricultural products.

2. Brief Description of the Prior Art

Driven by the potential for personalized medicine applications, the cost of sequencing DNA has seen a million-fold decrease in the last seven years. Whereas the cost of the first human genome was several billion dollars, the current cost is approaching one thousand. These advances were driven by improvements in DNA sequencing technologies that make possible to sequence millions of short DNA fragments in parallel. Although researchers working on a very small number of model species such as humans have realized the potential of this revolution, a large number of researchers studying the genetic diversity of the more than one million non-model species (such as crop pests) have yet to take advantage of the technological revolution. Since these researchers need to sequence many diverse species for a small fraction of the genome (100-500 genes), they require technologies that enable enrichment of homologous genomic DNA in a high throughput fashion. DNA samples must be prepared for sequencing through a process called library preparation. This involves ligation of common adapters onto the target DNA. For studies involving large numbers of samples (e.g.. 100s or 1000s), this is the cost-prohibitive step (˜$20-$80 per sample). This process can also be very time consuming, requiring up to one week per 100 samples.

Hybrid enrichment (e.g. whole-exome sequence capture), one technique for enriching genomic DNA for target regions of interest, involves the use of short fragments of DNA as probes to isolate genomic fragments of interest. The probes are typically designed based on preexisting genomes. Unfortunately, the available hybrid enrichment tools are limited to model species and can only be used for within species applications since the genes targeted typically vary too greatly for probes to be useful across species. Because efficient methods of target enrichment are lacking for non-model species, thousands of researchers are still utilizing antiquated sequencing techniques to study the diversity of living species.

An approach relatively similar to anchored hybrid enrichment has been disclosed in Golan et al., Weighted pooling—practical and cost-effective techniques for pooled high-throughput sequencing, Bioinformatics, Vol. 28, pp. i197-i206 (2012), which is incorporated herein by reference. However, this approach works only with one species (human), rather than being applicable across species or relying on differences among species.

Accordingly, what is needed is a methodology for high-throughput sequencing across- and within-species targeting full-length, rapidly-evolving genes on a short timescale. However, in view of the art considered as a whole at the time the present invention was made, it was not obvious to those of ordinary skill in the field of this invention how the shortcomings of the prior art could be overcome.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF DESCRIPTION OF THE DRAWINGS

For a fuller understanding of the invention, reference should be made to the following detailed description, taken in connection with the accompanying drawings, in which:

FIG. 1 is a graphical illustration of multi-anchors. Probes can be positioned in multiple, nearby conservation peaks in order to allow full-length genes to be obtained using the anchored hybrid enrichment approach. Here, sequence conservation of a gene is shown for 10 species of amniotes. The positions of probes are shown using horizontal lines on the top portion of the figure.

FIG. 2 depicts the workflow of MetaPrep, as compared to the workflow of the conventional art.

FIG. 3 is a chart illustrating results of Anonymous MetaPrep, in particular the percent of reads mapping for each pairwise combination of species in the Anonymous MetaPrep experiment. For most of the species, >90% of the reads could be accurately mapped within species (see diagonal values), suggesting that Anonymous MetaPrep is an efficient way to collect large quantities of population genetic data. Species with lower values were closely related species of fish. Anonymous MetaPrep was tested by pooling 14 different species in each of 81 different pools. Reads were mapped to the reference sequences that were used for probe design.

FIG. 4 illustrates results of Anchored MetaPrep, in particular data quality and the empirically-determined relationship between the pairwise sequence divergence, and the % of bad sequences produced, which is improved with the kmer blocking (labeled as Improvement A) approach and the contamination filter (labeled as Improvement B) approach. Anchored MetaPrep was tested by pooling samples across species in pools of either 5, 10 or 20 species. Three (3) different analysis procedures were employed: standard assembler, kmer blocking (labeled as Improvement A), and the contamination filter (labeled as Improvement B).

FIG. 5 results of Anchored MetaPrep, in particular data quality and the empirically-determined relationship between the pairwise sequence divergence and the number of good loci recovered.

### DETAILED DESCRIPTION OF THE PREFERRED EMBODIMENT

In the following detailed description of the preferred embodiments, reference is made to the accompanying drawings, which form a part thereof, and within which are shown by way of illustration specific embodiments by which the invention may be practiced. It is to be understood that other embodiments may be utilized and structural changes may be made without departing from the scope of the invention.

The current invention is based, in part, on anchored hybrid enrichment as disclosed in U.S. patent application Ser. No. 13/749,204 (the '204 application), which is incorporated herein by reference. In an embodiment, this anchored hybrid enrichment methodology can be utilized in the field of agricultural products, for example including, but not limited to, crop production, pesticide development, crop trait development, and pest control.

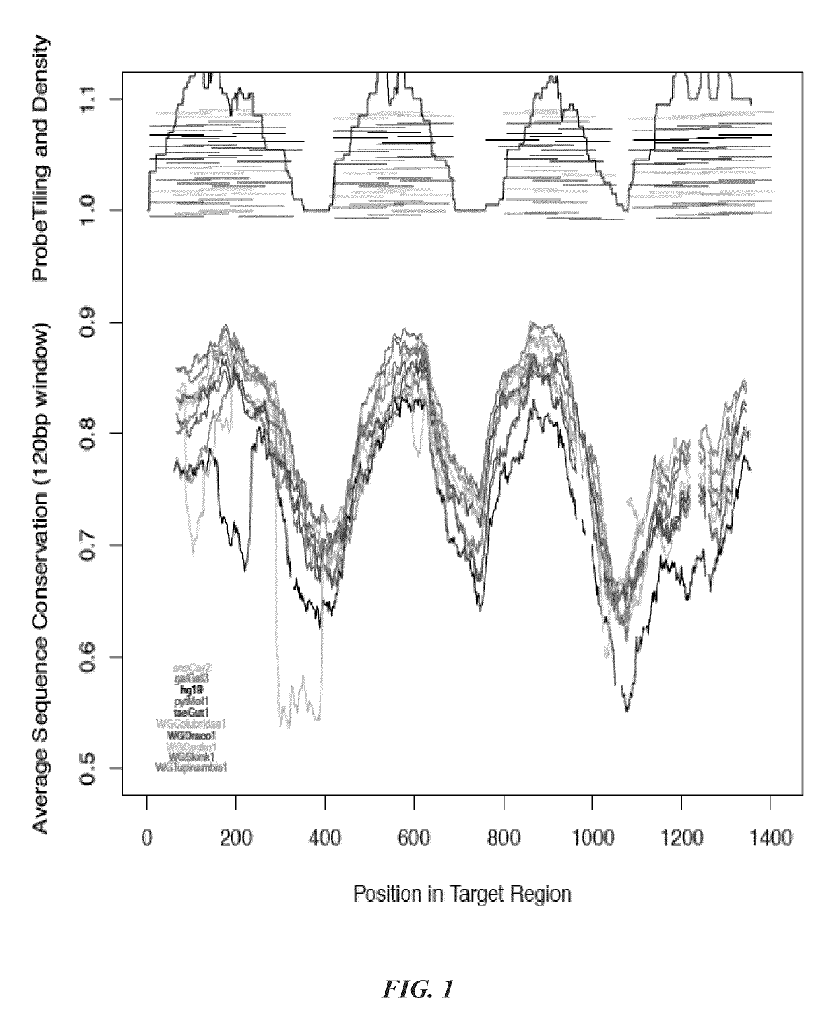

A couple of issues are contemplated when attempting to apply anchored hybrid enrichment to facilitate agricultural production. First, for agricultural research, studying the genetic variation in full-length target genes of agricultural interest (e.g., targets of insecticides) is of particular interest. Contrastingly, anchored hybrid enrichment typically is designed to target short regions of the genome that are not necessarily in target genes. However, it was discovered that the large majority of genes contain several conserved regions separated by less conserved regions. These conserved regions could be used as multi-anchors that would allow full-length genes to be targeted using the current approach. This can be seen in FIG. 1. Thus, this issue is resolved.

A second issue that is contemplated is that for agricultural research, there is a need to study the spread of introduced crop pests, for example, to sequence genes in the pest species that are evolving rapidly enough to provide information about the invasions that are happening on a short timescale. Contrastingly, due to the reliance on conserved regions with the anchored hybrid enrichment methodology, the type of genes targeted by the methodology would evolve too slowly to provide signal on short timescales. However, the methodology can be adapted for application to short timescales in the following way. Rather than targeting conserved regions, which evolve too slowly to be useful on short timescales, anonymous regions evolving much more rapidly can be targeted. These anonymous regions can be obtained for probe design using a small amount of initial genomic sequence data collected at the beginning of a study. Using a kit containing these probes, many individuals can be sequenced for the same set of rapidly evolving genes. Thus, this issue is also resolved.

In an embodiment, the current invention is a method that potentially allows dozens of samples to be pooled before library preparation, thereby reducing the cost of reagents and labor by a factor of 10 or more (to $2-$8 per sample or lower). The method also increases the rate at which samples can be processed by a similar magnitude (potentially thousands per week). A novel feature is the fact that samples are pooled prior to library preparation (instead of after library preparation as in the conventional art). The method relies on the ability to sort out the sequencing reads by individual after sequencing, which is possible if the pooled samples are of sufficient evolutionary divergence and/or if reference sequences for the species are available or can be obtained using individually indexed control samples. Diverse samples from within a project and/or across projects could potentially be sequenced using the current invention. Genome size is one factor that will potentially limit the number of samples that can be sequenced using the current methodology since a sufficient number of copies of each genome must be represented in the pool in order to avoid problems with PCR duplicates during library preparation. These and other important objects, advantages, and features of the invention will become clear as this disclosure proceeds.

In another embodiment, the current invention is a novel approach to target enrichment, for example anchored enrichment. The method utilizes highly conserved elements and anchors useful for capturing adjacent nonconserved regions (the '204 application; Lemmon et al. 2012). A small number of vertebrate genome sequences were utilized to develop a molecular toolkit that can be used to extract the same set of genes from any vertebrate species. The technique developed reduces the cost of collecting genetic data for studying biodiversity by a factor of 100. One of the most appealing aspect of this new approach is that it can be immediately applied to any species of vertebrate. Additional work is needed to extend this work to species valuable in agricultural markets (crop species, pest species, etc.), which is contemplated by this application.

The hybrid enrichment technology is superior to currently-used methods of assessing genetic variation across species for several reasons. First, currently used methods require substantial amounts of effort since each gene must be sequenced separately for each individual. The hybrid enrichment approach allows for simultaneous sequencing of hundreds of genes for hundreds of species simultaneously, thereby increasing the rate at which new genetic variations can be discovered by several orders of magnitude. Second, hybrid enrichment technology allows genes from new species to be sequenced without costly tool development (e.g., PCR primer design, etc.).

An example from agriculture will serve to demonstrate a new methodology related to the hybrid enrichment technology. Researchers working for BAYER are developing insecticides that target specific insect proteins. However, because these target proteins vary substantially across the insect species, the insecticides are only effective for a few species. An embodiment of the novel approach would allow the researchers developing the insecticides to obtain the gene sequences (and corresponding protein sequences) from any insect species. These sequences would be of great value because the insecticides could be engineered to more effectively target the proteins of the various insect species.

Additionally, the new method of assessing variation within species promises to be superior to currently used methods in at least three ways. First, currently used methods (named Rad-tag) produce data matrices that have large amounts of missing cells (i.e., many genes for many samples are missing). This negatively affects downstream analyses since genes not sequenced for all of the individuals in the study are typically thrown out. The current methodology promises to provide much more complete data matrices since hybrid enrichment technology is less sensitive to across-individual variation than Rad-tag technologies. Second, currently-used methods are not flexible with respect to the size of the genes that are sequenced. The novel methodology can be easily adjusted to meet the end users' needs. Finally, currently-used methods are static with respect to the set of genes that are obtained. In contrast, the current methodology can be adjusted in an iterative fashion to improve the genomic targets between subsequent studies and therefore increase the efficiency of the studies through time.

### EXAMPLE 1

In order to assess the potential of the anchored hybrid enrichment methodology, based in part on the methodology disclosed in the '204 application, to be applied outside of vertebrates, a probe design workshop was designed for biologists working on insects. Twelve (12) participants working on different insect groups undertook the workshop with large amounts of unpublished data sufficient to design an enrichment probe kit for insects. It was determined that the application of this anchored hybrid enrichment technology is feasible, thus suggesting the application of this technology to pest control and crop production.

### EXAMPLE 2

In an embodiment, preliminary calculations have shown that the current invention can decrease the cost of doing population genetics by a factor of >10. Additionally, the current invention can increase the sample throughput by a factor of >10. The methodology is quite effective for processing of a large number of projects simultaneously.

Potential Applications

Several projects will be undertaken related to agricultural product development to further assess the effectiveness of the current methodology in the agriculture market, in particular crop trait improvement, native pest control, and introduced pest prevention.

Crop trait improvement: The goal is to assess variation in corn strains. Using genomic coordinates for target genes provided by MONSANTO, hybrid enrichment probes will be designed and will be useful to obtain these genes in any strain of corn. Once the probe kit is produced by AGILENT, libraries will be prepared from DNA samples provided by MONSANTO (representing 96 different corn strains), DNA for the target regions will be enriched, enriched samples will be sequenced at HUDSON ALPHA (PE 100 bp), and the results will be analyzed in consultation with MONSANTO. Gene sequences will be estimated for 96 strains using sequence data. Products that result from this project may include (1) a probe design useful for isolating economically important genes, and (2) gene sequences for important corn genes for several strains of corn.

It is expected that the probe design for the corn genes should be successful. The enrichment efficiency may be lower than typical due to the somewhat large size of the corn genome (2.5 billion base pairs). However, this methodology of probe design has been successful with frog samples with larger genomes (up to 6 billion base pairs). Problems resulting from the larger genome size will be detected during analysis of the sequence data.

Native pest control: The goal is to survey genetic variation in insecticide targets. Using

DNA sequences for insecticide targets provided by BAYER CROPSCIENCE, hybrid enrichment probes will be designed for these targets. As many insect lineages as possible (likely ˜40) will be incorporated, given the constraints imposed by the limited number of probes in the AGILENT probe kit. Libraries will then be prepared from 96 DNA samples (i.e., 96 insect species) provided by BAYER, and sequenced at HUDSON ALPHA (PE 100 bp). Gene sequences corresponding to each of the 96 samples (insects) will then be reconstructed from the raw sequence data. Products that result from this project may include (1) a probe design useful for isolating insect genes targeted by insecticides, and (2) gene sequences for insecticide targets in 96 insect species.

It is expected that efficient probes can be designed for the insecticide target genes. The anchored hybrid enrichment probes are expected to be successful since these probes have been proven successful with vertebrates, and insects have smaller genome sizes than vertebrates. Efficiency of the probe sets can be assessed during analysis of the sequence data.

Introduced pest prevention: The goal is to test the within-species enrichment method. DNA samples from 96 Gypsy moths across their North American range will be provided by collaborators working on insects. For one of the samples, a library will be prepared and enriched, the enriched library will be sequenced at HUDSON ALPHA (PE 100 bp), and raw genomic sequence data/reads will be collected. Probes for 5000 genes will be designed from the raw sequence data. Libraries will then be prepared for the remaining Gypsy moth individuals and enriched using the probes. The enriched libraries will be sequenced at HUDSON ALPHA (PE 100 bp). After sequencing of the enriched libraries, raw sequence data will be used to estimate and reconstruct the sequences of the anonymous genes of each individual. Using these sequences, the invasion history will be reconstructed using the program PhyloMapper (Lemmon and Lemmon 2008). Products that result from this project may include (1) a probe design useful for estimating the invasion history of Gypsy moths, (2) 5,000 gene sequences for 96 Gypsy moth individuals, and (3) an estimate of invasion history for Gypsy moths in the United States.

It is expected that these probe designs will be successful, as enrichment will be performed for samples within a species. Though there is a possibility that anonymous loci (despite being more variable than the typical anchored loci) may not be variable enough to accurately reconstruct the invasion history, this is not expected. Any issues that arise can be detected during analysis of the sequence data.

MetaPrep Experimentation

a. Applications of the MetaPrep method

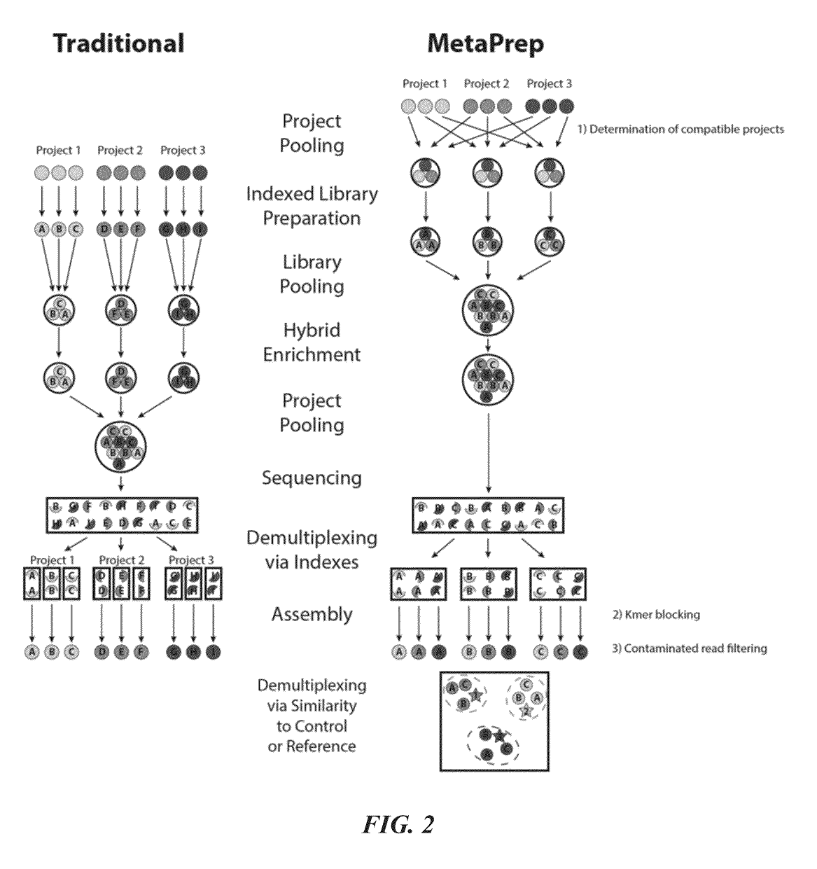

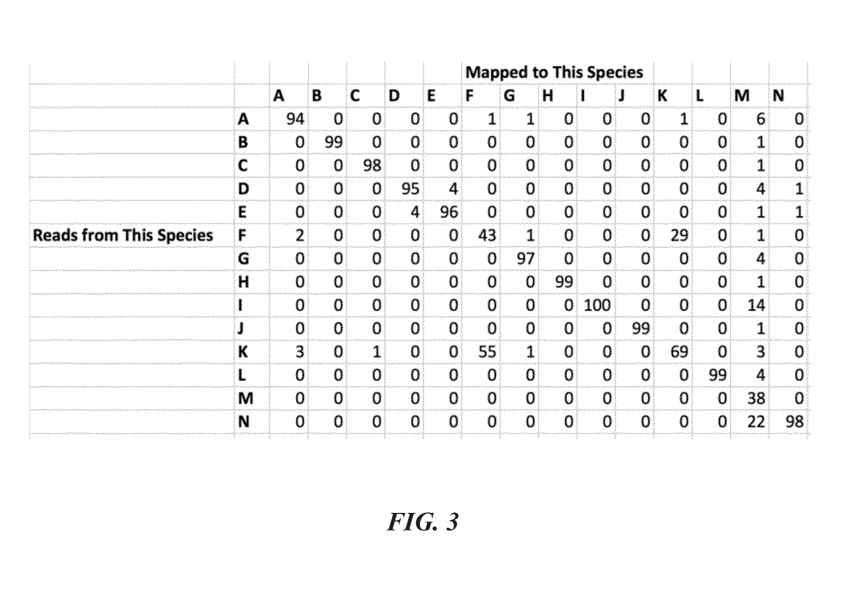

Two applications of the MetaPrep technology have been explored, the workflow of which can be seen in FIG. 2. The first application, termed “Anonymous MetaPrep” (results seen in FIG. 3), is the isolation of genomic regions that are different for each species in the MetaPrep pool. This approach is useful for efficiently obtaining large quantities of data at the population genetic timescale. This situation was tested in an experiment in which it was attempted to simultaneously enrich 1000 genomic regions (˜600 nucleotides each) for each of fourteen different species (7 vertebrates and 7 invertebrates). Each metaprep pool contained one individual from each of the 14 species. Data were collected for a total of 81 MetaPrep pools.

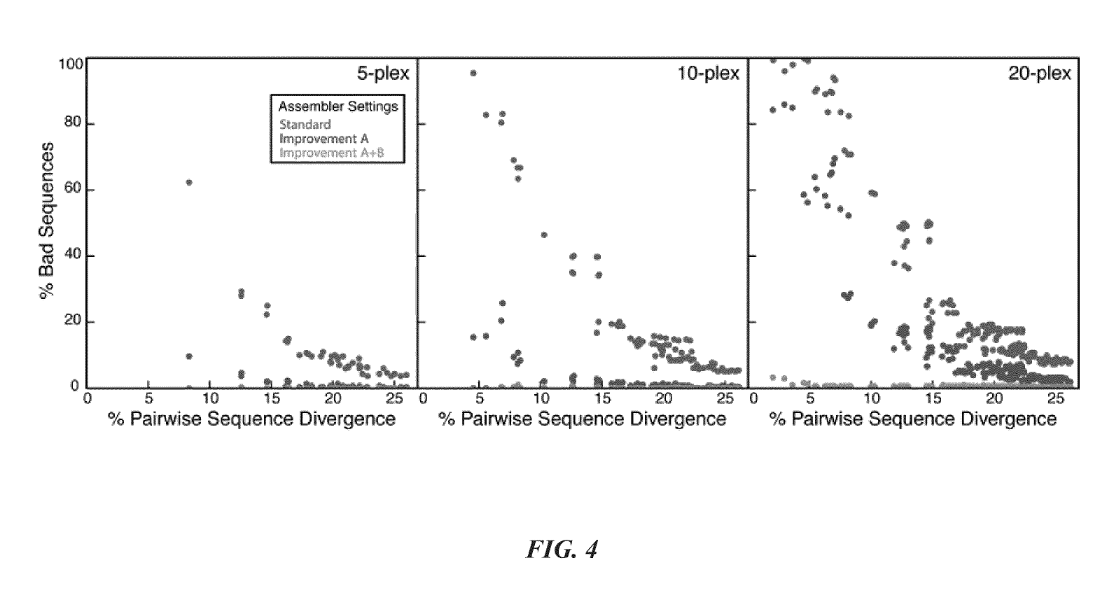

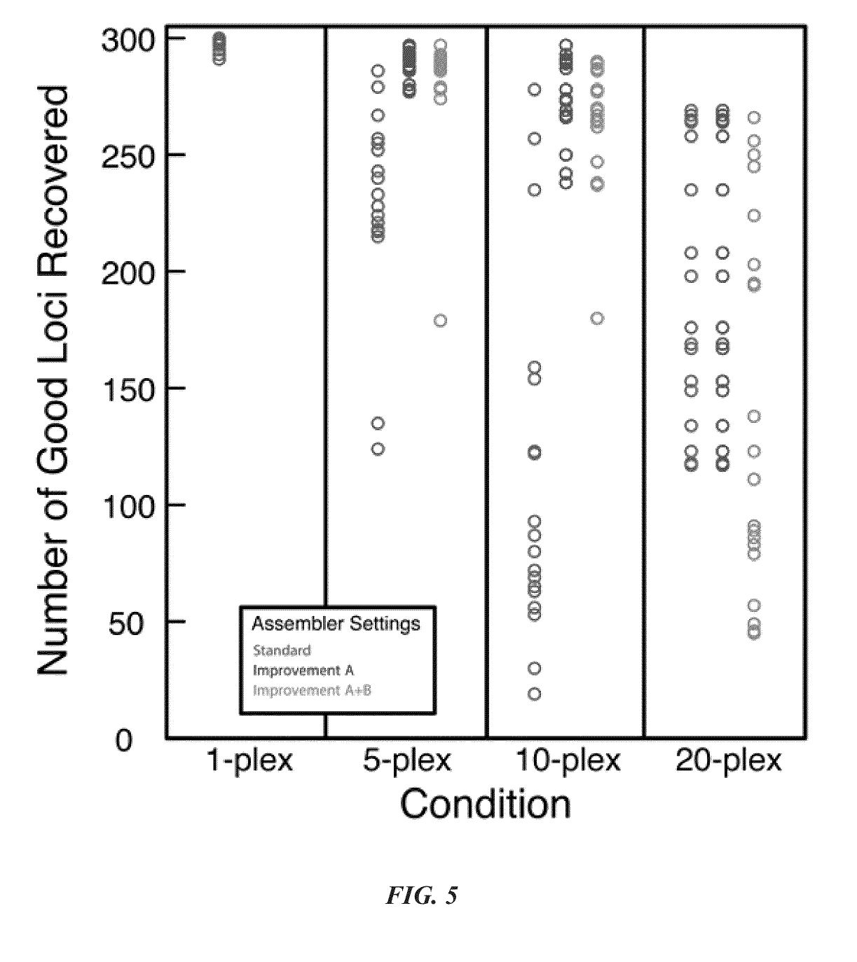

The second application, termed “Anchored MetaPrep” (results seen in FIGS. 4-5), is the isolation of genomic regions that are the same (homologous) for the species in the MetaPrep pool. This approach is useful for efficiently obtaining large quantities of data at phylogenetic timescales (deeper in time than population genetic timescale). This situation was tested in an experiment in which it was attempted to simultaneously enriched the same 300 genomic regions (˜1500 nucleotides each) for up to 20 species (all Amniotes).

b. Identification of Compatible Species/Loci

The first step of a MetaPrep study is the selection of loci and species that will be involved. In order for useful data to be obtained, the genomic region sequences for the species pooled must be sufficiently different to allow sorting of the reads during the post-sequencing assembly steps.

Locus selection for Anonymous MetaPrep involves 1) low-coverage (≧1×) genome sequencing of each of the species, 2) identification sequence reads from low-copy regions of the genome, and 3) selection of low-copy reads that do not have homologous reads from other species. Steps 2 and 3 can be performed by constructing kmer (e.g., 20-mer) databases and counting the number of genomic sequencing reads (both within and across species) that contain each 20 mer. Reads containing 20 mers found in large numbers (i.e., 10 times the sequencing depth) of reads within species are disqualified in step 2. Reads containing kmers with any significant number of reads (e.g. 1 or more) from another species can also be disqualified. Note that reads can also be filtered to contain only those that will allow capture efficiency to be optimized. For example, reads containing repetitive elements or imbalanced nucleotide composition may be removed. The set of species may also be adjusted in order to remove closely relate species that have a high proportion of across-species kmer matches. Once candidate loci are selected, probes can be designed from a set number of the reads (e.g., 1000 per species) that passed the filters. Enrichment kits containing reads from all of the species is then ordered.

Locus selection for Anchored MetaPrep involves 1) identification of homologous genomic regions across the species, 2) analysis of similarity across species to identify genomic regions with sufficient differences to allow downstream read sorting, and 3) identification of species that should be removed because their sequences are too similar to another species in the experiment. The genomic regions can be selected from those already utilized in standard (non-MetaPrepped) Anchored Enrichment. Similarity across species can be assessed by comparing species sequences pairwise, and identifying the length of the largest stretch of identical bases. Loci containing one or more identical stretches longer than some threshold (e.g. 60) can be identified as potentially problematic and removed from further consideration. Likewise, species pairs with a substantial number (e.g. >50%) of loci containing identical stretches longer than the threshold may also be removed.

c. Kmer Blocking

Prior to assembly, kmers identified as being problematic during locus selection (i.e., those found in more than one species) can be stored in a blacklist database. Kmer-based assembly can then proceed normally but without use of these blacklisted kmers. Genomic regions containing the blacklisted kmers will not be assembled directly (i.e. reads will not be mapped to the region using the blacklisted kmers) but instead may be assembled using nearby kmers that are not blacklisted. The kmer blocking approach reduces dramatically the levels across species contamination as seen in FIG. 4.

d. Contamination Filter

After assembly, consensus sequences can be analyzed for the occurrence of ambiguous base calls (i.e., not ‘A’, ‘T’, ‘C’, or ‘G’). Sequences with levels of ambiguous base calls higher than expected from biological causes (i.e., heterozygosity) can be removed from further analysis, thereby improving the quality of the final sequence list (FIG. 4).

### REFERENCES

Lemmon, A. R., S. Emme and E. C. Lemmon. 2012. Anchored hybrid enrichment for massively high-throughput phylogenetics. Systematic Biology. 61: 721-744.

Lemmon, A. R., and E. M. Lemmon. 2008. A likelihood framework for estimating phylogeographic history on a continuous landscape. Systematic Biology. 57: 544-561.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A method of generating cross-species genetic profiles using solution-based sequence capture, target enrichment probes to reconstruct a phylogenetic tree at a range of phylogenetic timescales in order to develop a pesticide, comprising the steps of:
sequencing a first genetic fragment acquired from a first species selected from a broader group, said first genetic fragment including a first conserved region and a first non-conserved region, said first conserved region coding for a first allele;
sequencing a second genetic fragment acquired from a second species selected from said broader group, said second genetic fragment including a second conserved region and a second non-conserved region, said second conserved region coding for a second allele homologous to said first allele, said first species and said second species selected from said broader group;
establishing a conservation standard and a uniqueness standard, said first conserved region and said second conserved region meeting said conservation standard and said uniqueness standard;
designing said solution-based sequence capture, target enrichment probes from said first genetic fragment and said second genetic fragment, such that said first conserved region and said second conserved region of said probes are respectively flanked by said first non-conserved region and said second non-conserved region, said first non-conserved region disposed adjacent to said first conserved region and said second non-conserved region disposed adjacent to said second conserved region;
preparing a library containing said target enrichment probes;
applying said library to a targeted locus;
sequencing said targeted locus as a result of a successful capture of said targeted locus, whereby sequence capture using said first and second conserved regions permits capture of large numbers of loci model species and highly-divergent non-model species, generation of said loci useful at said range of phylogenetic timescales, and production of said loci capable of resolving said phylogenetic tree despite gene tree discordance; and
developing said pesticide by targeting sequences resulting from said sequencing of said targeted locus.
